(19) United States
(12) Patent Application Publication
(10) Pub. No.: US 2024/0164746 A1
(43) Pub. Date: May 23, 2024

(54) ULTRASONIC DIAGNOSTIC SYSTEM

(71) Applicants: FUJI CORPORATION, Chiryu (JP); ASAHI INTECC CO., LTD., Seto-shi (JP)

(72) Inventors: Naofumi YOSHIDA, Obu-shi (JP); Yasuhiro YAMASHITA, Toyoake-shi (JP); Shinya KONDO, Okazaki-shi (JP); Makoto NISHIUCHI, Seto-shi (JP); Manabu SHIMOGAMI, Seto-shi (JP); Fumiyoshi OSHIMA, Seto-shi (JP)

(73) Assignees: FUJI CORPORATION, Chiryu (JP); ASAHI INTECC CO., LTD., Seto-shi (JP)

(21) Appl. No.: 18/549,203

(22) PCT Filed: Mar. 17, 2021
(86) PCT No.: PCT/JP2021/010902

§ 371 Date: Sep. 6, 2023

(51) Int. Cl. A61B 8/00; A61B 34/20; A61B 34/30

(52) U.S. Cl. CPC A61B 8/4218; A61B 8/4245; A61B 8/4405; A61B 8/543; A61B 34/20; A61B 34/30; A61B 2034/2065; A61B 2034/305

(57) ABSTRACT

An ultrasonic diagnostic system includes an ultrasonic diagnostic device having a probe, a moving device configured to move the probe, and a control device. When ultrasonic diagnosis is performed on a subject, the control device controls the moving device such that the probe is pressed against a body surface of the subject, and in a case where a specific pattern is designated as a movement pattern of the probe, controls the moving device such that the pressing of the probe on the body surface is canceled or alleviated, then controls the moving device such that the probe is moved with the specific pattern, and when the movement with the specific pattern is ended, performs a specific control for controlling the moving device such that the probe is pressed against the body surface.

### TECHNICAL FIELD

The present description discloses an ultrasonic diagnostic system.

### BACKGROUND ART

Conventionally, as such an ultrasonic diagnostic system, one in which a treatment head including a diagnostic probe is attached to a tip end of a robot arm has been proposed (see Patent Literature 1, for example). In this system, a force sensor that detects the magnitude and direction of a force acting on the robot arm via the treatment head is provided, and when the diagnostic probe is pressed against a treatment target, a probe driving device is controlled so that the force detected by the force sensor is equal to or less than a threshold value.

### PATENT LITERATURE

Patent Literature 1: JP-A-2019-165839

### SUMMARY OF THE INVENTION

### Technical Problem

Incidentally, in a case where a probe is moved by a moving device in a specific pattern different from a normal movement pattern, depending on the specific pattern, if the probe is moved in a state of being pressed against a subject, a large burden may be placed on the body of the subject.

It is a main object of the present disclosure to provide a technique for moving a probe by a moving device in ultrasonic diagnosis, which further reduces a burden on a subject when moving the probe in a specific pattern.

### Solution to Problem

The present disclosure employs the following means in order to achieve the main object described above.

An ultrasonic diagnostic system of the present disclosure includes an ultrasonic diagnostic device having a probe, a moving device configured to move the probe, and a control device configured to perform a specific pattern control for controlling the moving device such that the probe is pressed against a body surface of a subject, or such that the pressing of the probe against the body surface is canceled or alleviated when ultrasonic diagnosis is performed on the subject.

In the ultrasonic diagnostic system of the present disclosure, the probe is moved by the moving device during ultrasonic diagnosis. The ultrasonic diagnostic system can reduce a burden on the subject during ultrasonic diagnosis.

### BRIEF DESCRIPTION OF DRAWINGS

FIG. 1 is an appearance perspective view of an ultrasonic diagnostic system of a first embodiment.

FIG. 2 is a side view of a robot.

FIG. 3 is a block diagram illustrating an electrical connection relationship among the robot, a control device, and an ultrasonic diagnostic device according to the first embodiment.

FIG. 4 is a flowchart illustrating an example of ultrasonic diagnostic processing.

FIG. 5 is a flowchart illustrating an example of specific operation processing.

FIG. 6 is a block diagram illustrating an electrical connection relationship between a robot, a control device, and an ultrasonic diagnostic device according to a second embodiment.

FIG. 7 is a flowchart illustrating ultrasonic diagnostic processing according to the second embodiment.

FIG. 8 is a flowchart illustrating specific operation processing of the second embodiment.

FIG. 9 is a flowchart illustrating specific operation processing of a third embodiment.

FIG. 10 is a flowchart illustrating specific operation processing of a fourth embodiment.

### DESCRIPTION OF EMBODIMENTS

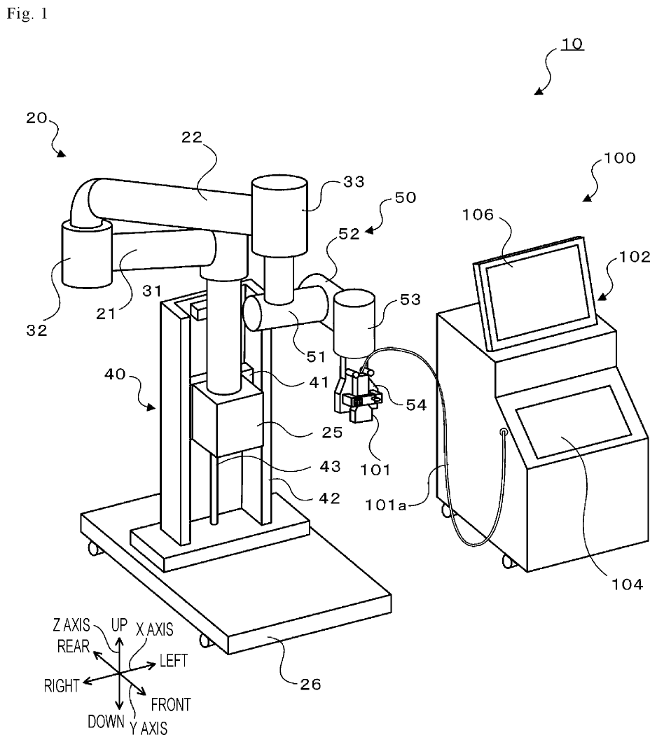

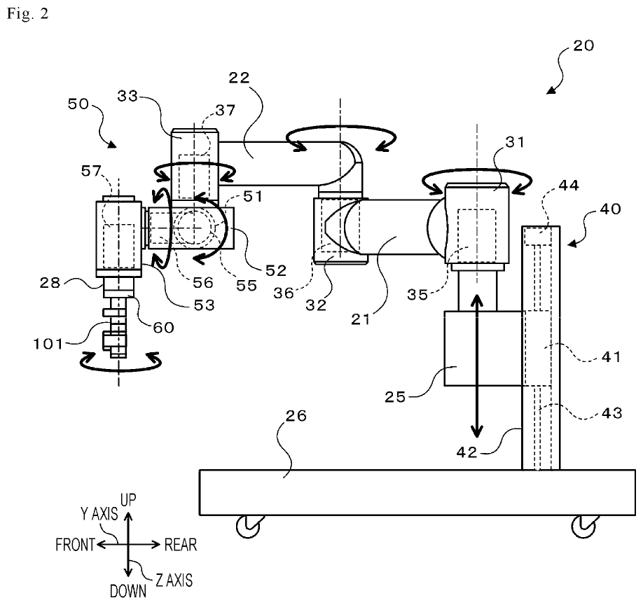

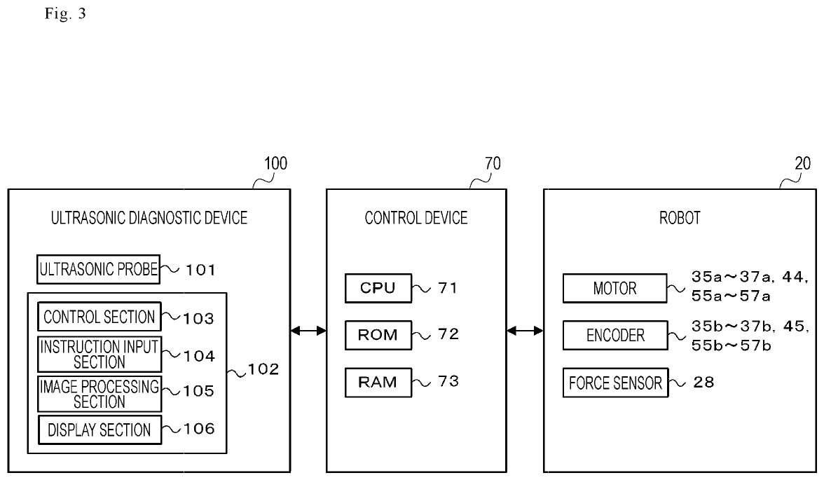

Next, an embodiment of the present disclosure will be described with reference to drawings. FIG. 1 is an appearance perspective view of an ultrasonic diagnostic system of a first embodiment. FIG. 2 is a side view of a robot. FIG. 3 is a block diagram illustrating an electrical connection relationship among the robot, a control device, and an ultrasonic diagnostic device according to the first embodiment. In FIG. 1, a left-right direction is an X axis, a front-rear direction is a Y-axis direction, and an up-down direction is a Z-axis direction.

Ultrasonic diagnostic system 10 of a first embodiment acquires an ultrasonic echo image by holding ultrasonic probe 101 at the fingertip of robot 20 and driving robot 20 automatically or remotely so that ultrasonic probe 101 is pressed against the body surface of a patient. Ultrasonic diagnostic system 10 is used, for example, in catheter treatment. An operator who operates a guide wire of a catheter can accurately pass a guide wire through an occlusion portion or a stenosis portion of a blood vessel by advancing the guide wire while recognizing a positional relationship between a tip end of the guide wire and the blood vessel from an ultrasonic echo image obtained by pressing ultrasonic probe 101 against the body surface of the patient.

As illustrated in FIGS. 1 to 3, ultrasonic diagnostic system 10 includes robot 20, force sensor 28, control device 70 that controls robot 20, and ultrasonic diagnostic device 100 including ultrasonic probe 101.

Ultrasonic diagnostic device 100 includes ultrasonic probe 101 and ultrasonic diagnostic device main body 102 connected to ultrasonic probe 101 via cable 101a. Ultrasonic diagnostic device main body 102 includes control section 103 that controls the entire device, instruction input section 104 configured to input an instruction to initiate diagnosis or the like, image processing section 105 that processes a received signal from ultrasonic probe 101 to generate an ultrasonic echo image, and display section 106 that displays the generated ultrasonic echo image.

As illustrated in FIG. 2, robot 20 includes first arm 21, second arm 22, base 25, base plate 26, first arm driving device 35, second arm driving device 36, orientation holding device 37, lifting and lowering device 40, three-axis rotation mechanism 50, and holder 60. First arm 21, second arm 22, and three-axis rotation mechanism 50 may be simply referred to as an arm.

The base end portion of first arm 21 is coupled to base 25 via first joint shaft 31 extending in the up-down direction (Z-axis direction). First arm driving device 35 includes motor 35a and encoder 35b. The rotation shaft of motor 35a is connected to first joint shaft 31 via a deceleration device (not illustrated). First arm driving device 35 causes first arm 21 to turn (pivot) along a horizontal plane (XY-plane) around first joint shaft 31 as a fulcrum by rotationally driving first joint shaft 31 by motor 35a. Encoder 35b is attached to the rotation shaft of motor 35a, and is configured as a rotation encoder that detects a rotational displacement amount of motor 35a.

The base end portion of second arm 22 is coupled to the tip end portion of first arm 21 via second joint shaft 32 extending in the up-down direction. Second arm driving device 36 includes motor 36a and encoder 36b. The rotation shaft of motor 36a is connected to second joint shaft 32 via a deceleration device (not illustrated). Second arm driving device 36 causes second arm 22 to turn (pivot) along a horizontal plane around second joint shaft 32 as a fulcrum by rotationally driving second joint shaft 32 by motor 36a. Encoder 36b is attached to the rotation shaft of motor 36a, and is configured as a rotation encoder that detects the rotational displacement amount of motor 36a.

Base 25 is provided so as to be lifted and lowered with respect to base plate 26 by lifting and lowering device 40 installed on base plate 26. As illustrated in FIGS. 1 and 2, lifting and lowering device 40 includes slider 41 fixed to base 25, guide member 42 fixed to base plate 26 and extending in the up-down direction to guide the movement of slider 41, ball screw shaft 43 (a lifting and lowering shaft) extending in the up-down direction and screwed into a ball screw nut (not illustrated) fixed to slider 41, motor 44 for rotationally driving ball screw shaft 43, and encoder 45 (see FIG. 3). Lifting and lowering device 40 causes base 25 fixed to slider 41 to move in the up-down direction along with guide member 42 by rotationally driving ball screw shaft 43 by motor 44. Encoder 45 is configured as a linear encoder that detects a position in the up-down direction (a lifting and lowering position) of slider 41 (base 25).

Three-axis rotation mechanism 50 is coupled to the tip end portion of second arm 22 via orientation holding shaft 33 extending in the up-down direction. Three-axis rotation mechanism 50 includes first rotation shaft 51, second rotation shaft 52, and third rotation shaft 53 that are orthogonal to each other, first rotation device 55 that rotates first rotation shaft 51, second rotation device 56 that rotates second rotation shaft 52, and third rotation device 57 that rotates third rotation shaft 53. First rotation shaft 51 is supported in an orientation orthogonal to orientation holding shaft 33. Second rotation shaft 52 is supported in an orientation orthogonal to first rotation shaft 51. Third rotation shaft 53 is supported in an orientation orthogonal to second rotation shaft 52. First rotation device 55 includes motor 55a for rotationally driving first rotation shaft 51, and encoder 55b attached to the rotation shaft of motor 55a to detect the rotational displacement amount of motor 55a. Second rotation device 56 includes motor 56a for rotationally driving second rotation shaft 52, and encoder 56b attached to the rotation shaft of motor 56a to detect the rotational displacement amount of motor 56a. Third rotation device 57 includes motor 57a for rotationally driving third rotation shaft 53, and encoder 57b attached to the rotation shaft of motor 57a to detect the rotational displacement amount of motor 57a. Holder 60 for holding ultrasonic probe 101 is attached to third rotation shaft 53. In the present embodiment, ultrasonic probe 101 is held by holder 60 so as to be positioned coaxially with third rotation shaft 53.

Robot 20 of the present embodiment can move ultrasonic probe 101 to any position in any orientation by a combination of the translational movement in three directions of the X-axis direction, the Y-axis direction, and the Z-axis direction by first arm driving device 35, second arm driving device 36, and lifting and lowering device 40, and the rotational movement in three directions of the X axis (pitching), the Y axis (rolling), and the Z axis (yawing) by three-axis rotation mechanism 50.

Orientation holding device 37 holds the orientation of three-axis rotation mechanism 50 (the direction of first rotation shaft 51) in a fixed direction regardless of the orientations of first arm 21 and second arm 22. Orientation holding device 37 includes motor 37a and encoder 37b. The rotation shaft of motor 37a is connected to orientation holding shaft 33 via a deceleration device (not illustrated). Orientation holding device 37 sets a target rotation angle of orientation holding shaft 33 based on the rotation angle of first joint shaft 31 and the rotation angle of second joint shaft 32 such that the axial direction of first rotation shaft 51 is always in the left-right direction (the X-axis direction), and drives to control motor 37a such that orientation holding shaft 33 is at the target rotation angle. As a result, the control of the translational movement in the three directions and the control of the rotational movement in the three directions can be independently performed to facilitate the control.

Force sensor 28 is attached to the tip end of the arm, and detects a force component acting in each axial direction of the X axis, the Y axis, and the Z axis as an external force acting on the arms, and a torque component acting around each axis.

As illustrated in FIG. 3, control device 70 is configured as a microprocessor centered on CPU 71, and includes ROM 72, RAM 73, an input/output port, and a communication port (not illustrated) in addition to CPU 71. The detection signal from force sensor 28, detection signals from respective encoders 35b, 36b, 37b, 45, 55b, 56b, 57b, and the like are input to control device 70 via input ports. In addition, a drive signal to each of motors 35a, 36a, 37a, 44, 55a, 56a, and 57a is output from control device 70 via output ports. Control device 70 communicates with control section 103 of ultrasonic diagnostic device 100 via a communication port to exchange data.

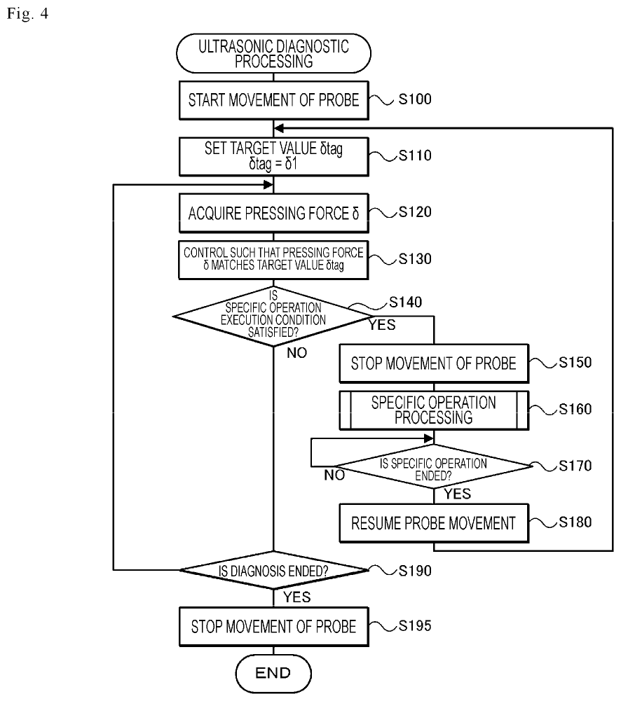

Next, an operation of ultrasonic diagnostic system 10 according to the present embodiment configured as described above will be described. FIG. 4 is a flowchart illustrating an example of ultrasonic diagnostic processing executed by CPU 71 of control device 70. This processing is executed, for example, when a user instructs instruction input section 104 to initiate a diagnosis.

When the ultrasonic diagnostic processing is executed, CPU 71 first drives and causes the corresponding motor of robot 20 to start the movement of ultrasonic probe 101 with respect to the patient (step S100). The movement of ultrasonic probe 101 is performed as follows. That is, CPU 71 determines a target position and a target orientation of an arm holding ultrasonic probe 101 according to a task program created in advance. Subsequently, CPU 71 sets a target rotation angle of first joint shaft 31, a target rotation angle of second joint shaft 32, a target rotation angle of orientation holding shaft 33, a target lifting and lowering position of base 25, a target rotation angle of first rotation shaft 51, a target rotation angle of second rotation shaft 52, and a target rotation angle of third rotation shaft 53, respectively, for moving the arm to a target position in a target orientation. Then, CPU 71 controls the corresponding motor such that the rotation angle or the lifting and lowering position detected by respective encoders 35b, 36b, 37b, 45, 55b, 56b, and 57b coincides with the corresponding target rotation angle or target lifting and lowering position. When ultrasonic diagnostic processing is applied to catheter treatment, CPU 71 controls robot 20 by setting the movement route and the movement speed such that ultrasonic probe 101 moves in accordance with the progress of the guide wire of a catheter while aligning the scanning direction of ultrasonic probe 101 with the center axial direction (length direction) of the blood vessel of the patient.

When the movement of ultrasonic probe 101 is started, CPU 71 sets predetermined value δ1 as target value δtag of the pressing force when ultrasonic probe 101 is pushed into the body surface of the patient (step S110). Next, CPU 71 acquires pressing force δ of ultrasonic probe 101 from force sensor 28 (step S120), and controls each of motors 35a, 36a, 37a, 44, 55a, 56a, 57a of robot 20 such that the pressing force δ matches target value δtag (step S130). Here, predetermined value δ1 can be determined in advance by experiments or the like so that the distance between ultrasonic probe 101 and the blood vessel as a diagnosis target falls within the effective depth of the ultrasonic wave within a range in which the patient does not feel pain or discomfort. By controlling the pressing of ultrasonic probe 101 by setting predetermined value δ1 as target value δtag, ultrasonic probe 101 can be pressed against the patient by an appropriate force, and a good ultrasonic echo image can be acquired without applying an excessive burden to the patient.

Next, CPU 71 determines whether the execution condition of the operation (specific operation) with a specific pattern is satisfied (step S140). The execution condition of the specific operation may be satisfied at a predetermined timing, or may be satisfied when the user instructs instruction input section 104 to execute a specific operation. When it is determined that the execution condition of the specific operation is not satisfied, CPU 71 determines whether the current diagnosis is ended (step S190). This determination may be made based on whether ultrasonic probe 101 has reached a predetermined position, or may be made based on whether the user instructs instruction input section 104 to end the diagnosis. When it is determined that the current diagnosis is not ended, CPU 71 returns to step S120, and continues the movement of ultrasonic probe 101 while maintaining pressing force δ at target value Stag. On the other hand, when it is determined that the current diagnosis is ended, CPU 71 stops the movement of ultrasonic probe 101 (step S195), and ends the ultrasonic diagnostic processing.

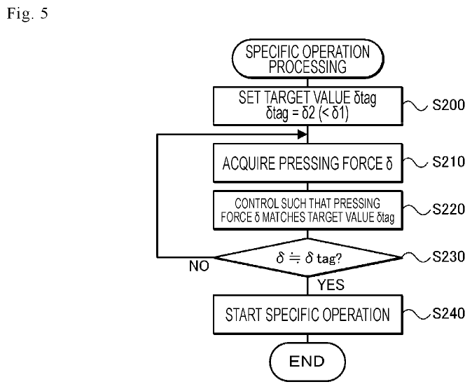

In step S140, when it is determined that the execution condition of the specific operation is satisfied, CPU 71 temporarily stops the movement of ultrasonic probe 101 (step S150) and executes specific operation processing (step S160). Here, the specific operation is an operation in which the burden on the patient is excessive when ultrasonic probe 101 is performed in a state of being pressed against the body surface of the patient, and examples thereof include a rotation operation for rotating ultrasonic probe 101 by 90 degrees about the axis, a movement operation for moving ultrasonic probe 101 one time by a predetermined distance or more, and the like. The rotation operation is performed, for example, in catheter treatment, to change the orientation from a state in which ultrasonic probe 101 scans the blood vessel in the length direction to a state in which ultrasonic probe 101 scans the blood vessel in the width direction, and to inspect whether the guide wire of the catheter deviates from the center of the blood vessel from the acquired ultrasonic echo image of the cross section of the blood vessel in the width direction. In addition, the movement operation is performed, for example, to move to another diagnosis portion after the diagnosis of one diagnosis portion is ended when diagnosing multiple portions at one time. The specific operation processing will now be described in more detail. FIG. 5 is a flowchart illustrating an example of the specific operation processing executed by CPU 71.

In the specific operation processing, CPU 71 first sets predetermined value δ2 that is smaller than the above-described predetermined value δ1 to target value δtag of the pressing force (step S200). Subsequently, CPU 71 acquires pressing force δ of ultrasonic probe 101 from force sensor 28 (step S210), and controls the corresponding motor of robot 20 such that pressing force δ matches target value δtag (step S220). In this processing, the pressing of ultrasonic probe 101 is controlled by setting predetermined value δ2 smaller than normal to target value δtag of the pressing force such that the pressing of ultrasonic probe 101 is canceled or alleviated. In the present embodiment, predetermined value δ2 is determined, for example, in the vicinity of a value 0 such that the pressing of ultrasonic probe 101 is canceled. Then, CPU 71 determines whether pressing force δ matches target value Stag (step S230). When it is determined that pressing force δ does not match target value Stag, CPU 71 returns to step S210 to repeat the control for canceling or alleviating the pressing force, and when it is determined that pressing force δ matches target value Stag, starts the specific operation (step S240), and ends the specific operation processing. As described above, when the specific operation is performed in a state in which ultrasonic probe 101 is pressed against the body surface of the patient, the burden on the patient is excessively large, and therefore the burden on the patient can be reduced by canceling or alleviating the pressing of the ultrasonic probe 101 before the execution of the specific operation. As described above, in the catheter treatment, in a case where the rotation operation is performed as a specific operation and the blood vessel into which the guide wire is inserted is scanned in the width direction, and the deviation amount of the guide wire with respect to the center of the blood vessel is inspected from the acquired ultrasonic echo image, ultrasonic diagnostic device 100 may output a warning to display section 106 when the deviation amount exceeds a predetermined amount.

Returning to the ultrasonic diagnostic processing, when the specific operation is started, CPU 71 waits for the specific operation to be ended (step S170), resumes the movement of ultrasonic probe 101 (step S180), and returns to step S110. That is, CPU 71 returns target value Stag of the pressing force from predetermined value δ2 to predetermined value δ1, presses ultrasonic probe 101 against the body surface of the patient with target value δtag of predetermined value δ1, and resumes the movement of ultrasonic probe 101.

Here, a correspondence relationship between main elements of the embodiment and main elements of the present disclosure recited in claims will be described. That is, ultrasonic probe 101 of the present embodiment corresponds to the probe of the present disclosure, ultrasonic diagnostic device 100 corresponds to an ultrasonic diagnostic device, robot 20 corresponds to a moving device, and control device 70 corresponds to a control device. In addition, force sensor 28 corresponds to a force sensor.

Needless to say, the present disclosure is not limited to the embodiment that has been described heretofore in any way and may be implemented in various forms without departing from the technical scope of the present disclosure.

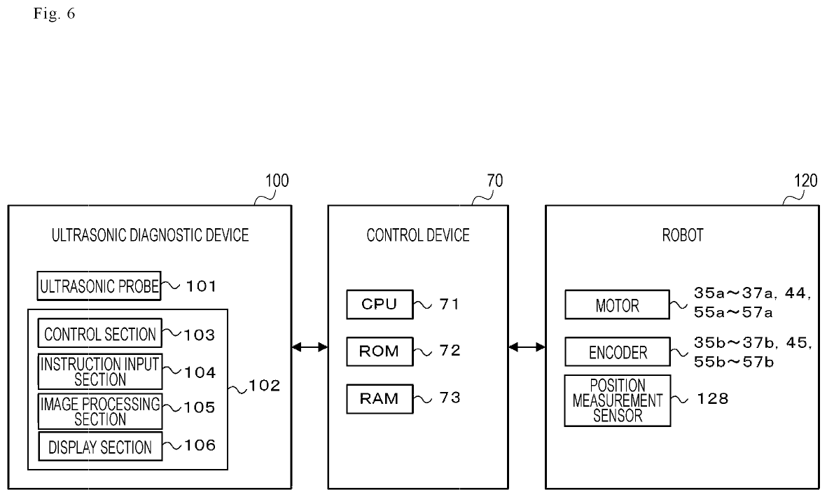

For example, in the above embodiment, when executing a specific operation, CPU 71 cancels or alleviates the pressing of ultrasonic probe 101 on the body surface of the patient by controlling the pressing force based on a signal from force sensor 28 before the execution. On the other hand, the ultrasonic diagnostic system of a second embodiment controls the position of ultrasonic probe 101 such that the pressing of ultrasonic probe 101 on the body surface of the patient is cancelled or alleviated. FIG. 6 is a block diagram illustrating an electrical connection relationship between a robot, a control device, and an ultrasonic diagnostic device according to a second embodiment. As illustrated, in the second embodiment, robot 120 includes position measurement sensor 128 in place of force sensor 28 as a sensor used for controlling the position of ultrasonic probe 101 when ultrasonic probe 101 is pressed against the body surface of the patient. Examples of position measurement sensor 128 may include a camera sensor, an eddy current type displacement sensor, a reflective laser displacement sensor, a capacitive displacement sensor, a magnetic induction type displacement sensor, and the like.

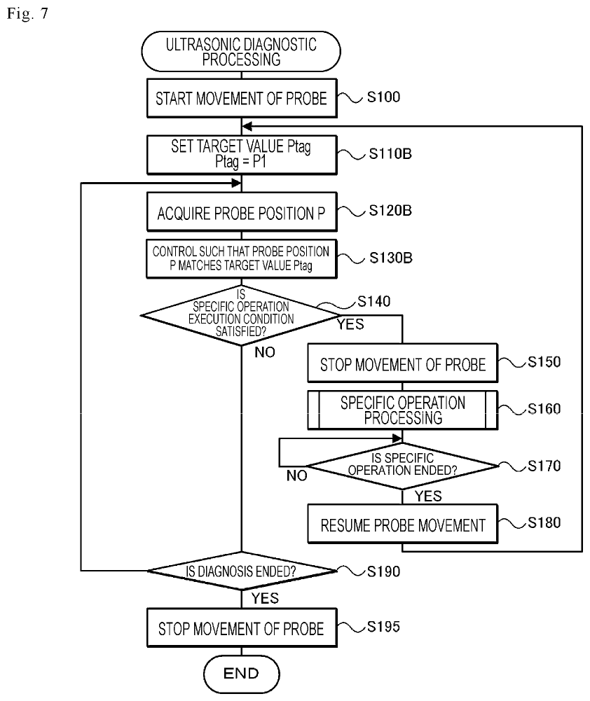

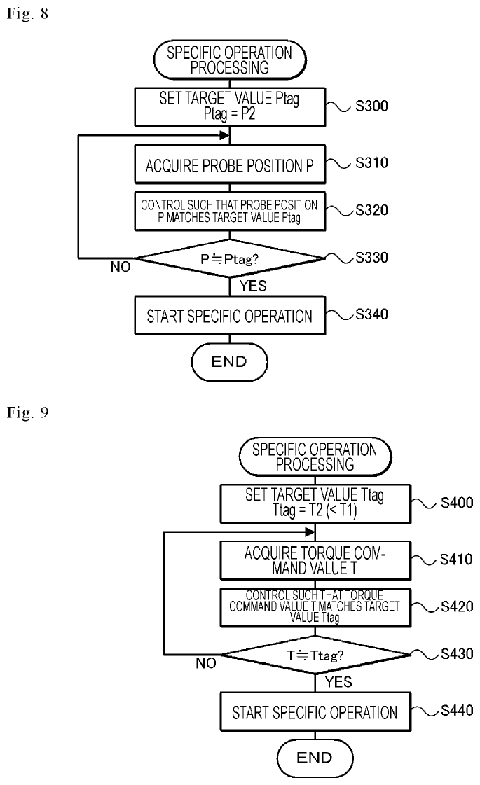

FIG. 7 is a flowchart illustrating ultrasonic diagnostic processing according to the second embodiment. In addition, FIG. 8 is a flowchart illustrating specific operation processing of the second embodiment. In the ultrasonic diagnostic processing in FIG. 7, the same processing as the ultrasonic diagnostic processing in FIG. 4 is denoted by the same step number, and repetitive descriptions thereof will be omitted.

In the ultrasonic diagnostic processing of the second embodiment, when the movement of ultrasonic probe 101 is started in step S100, CPU 71 sets position P1 as target value Ptag of the position (probe position) of ultrasonic probe 101 (step S110B). Here, when a camera sensor is used as position measurement sensor 128, CPU 71 images the body surface of the patient with the camera sensor in advance before diagnosis, and measures the position of the body surface as a reference position from the acquired captured image. Target value Ptag is determined as the position (position P1) of ultrasonic probe 101 when ultrasonic probe 101 is pushed into the reference position by a predetermined amount. Subsequently, CPU 71 acquires probe position P from position measurement sensor 128 (step S120B), and controls each of motors 35a, 36a, 37a, 44, 55a, 56a, and 57a such that acquired probe position P matches target value Ptag (step S130B). Then, when it is determined in step S140 that the execution condition of the specific operation is not satisfied and it is determined in step S190 that the diagnosis is not ended, CPU 71 returns to step S120B and continues the movement of ultrasonic probe 101 while maintaining probe position P at target value Ptag. On the other hand, when it is determined that the execution condition of the specific operation is satisfied in step S140, CPU 71 temporarily stops the movement of ultrasonic probe 101, then executes the specific operation processing, and when the specific operation is ended, resumes the movement of ultrasonic probe 101 (steps S150 to S180), and returns to step S110B.

In the specific operation processing of the second embodiment, CPU 71 sets position P2 as target value Ptag of the probe position (step S300). Position P2 is determined as a position spaced apart from the body surface of the patient by a predetermined distance or more than when ultrasonic probe 101 is pushed in with position P1 as target value Ptag in the ultrasonic diagnostic processing of FIG. 7. Next, CPU 71 acquires probe position P (step S310). Subsequently, CPU 71 controls each of motors 35a, 36a, 37a, 44, 55a, 56a, and 57a such that acquired probe position P matches target value Ptag (step S320). Then, CPU 71 then determines whether probe position P matches target value Ptag (step S330). When it is determined that probe position P does not match target value Ptag, CPU 71 returns to step S310 to repeat the processing, and when it is determined that probe position P matches target value Ptag, starts the specific operation (step S340), and ends the specific operation processing.

In the second embodiment, CPU 71 acquires probe position P from position measurement sensor 128, and controls the pressing of ultrasonic probe 101 based on acquired probe position P and reference position. However, CPU 71 may calculate arm position P by forward kinematics based on the rotation angle or the lifting and lowering position detected by encoders 35b, 36b, 37b, 45, 55b, 56b, and 57b of each joint shaft or each rotation shaft, and control the pressing of ultrasonic probe 101 based on calculated probe position P and the reference position.

The ultrasonic diagnostic system of a third embodiment controls the pressing torque of ultrasonic probe 101 to cancel or alleviate the pressing of ultrasonic probe 101 on the body surface of the patient. FIG. 9 is a flowchart illustrating specific operation processing of a third embodiment.

In the specific operation processing of the third embodiment, CPU 71 sets predetermined torque T2 as target value Ttag of the torque of the motor that outputs the pressing torque for pressing ultrasonic probe 101 against the body surface of the patient (step S400). Predetermined torque T2 is determined to be a torque that is approximately smaller than torque T1 in the pressing direction required when ultrasonic probe 101 is pressed against the body surface of the patient in the ultrasonic diagnostic processing. Next, CPU 71 acquires torque command value T of the motor outputting the torque in the pressing direction of ultrasonic probe 101 (step S410). Subsequently, CPU 71 controls the corresponding motor by reducing torque command value T such that acquired torque command value T matches target value Ttag (step S420). Then, CPU 71 then determines whether torque command value T matches target value Ptag (step S430). When it is determined that torque command value T does not match target value Ttag, CPU 71 returns to step S410 to repeat the processing, and when it is determined that torque command value T matches target value Ttag, executes the specific operation (step S440), and ends the specific operation processing.

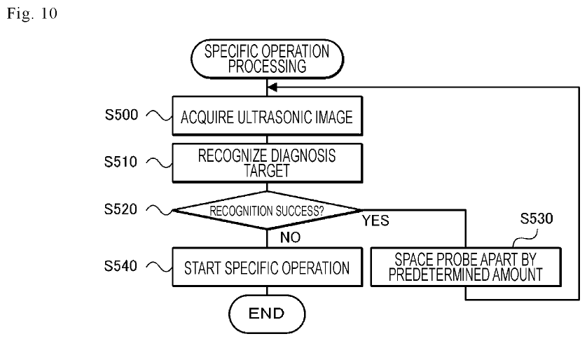

The ultrasonic system of a fourth embodiment cancels or alleviates the pressing of ultrasonic probe 101 on the body surface of the patient by spacing ultrasonic probe 101 apart from the patient until the blood vessel of the patient cannot be recognized by the ultrasonic echo image. FIG. 10 is a flowchart illustrating specific operation processing of a fourth embodiment.

In the specific operation processing of the fourth embodiment, CPU 71 acquires an ultrasonic echo image (step S500). Subsequently, CPU 71 performs image processing for recognizing a diagnosis target (blood vessel) from the acquired ultrasonic echo image (step S510), and determines whether the recognition is successful (step S520). The image processing can be performed, for example, by applying pattern matching to the acquired ultrasonic echo image. When it is determined that the recognition is successful, CPU 71 controls the corresponding motor such that ultrasonic probe 101 is spaced apart from the body surface of the patient by a predetermined amount (step S530), returns to step S500, and repeats the processing of steps S500 to S520. Then, when it is determined in step S520 that the recognition of the diagnosis target has failed in the course of the repetition of the processing, CPU 71 executes the specific operation (step S540) and ends the specific operation processing. When ultrasonic probe 101 is spaced from the body surface of the patient by the predetermined amount, the resolution of the acquired ultrasonic echo image gradually decreases. Accordingly, at the time when the diagnosis target (blood vessel) cannot be recognized from the ultrasonic echo image, CPU 71 can determine that ultrasonic probe 101 is sufficiently spaced from the body surface, and the pressing of ultrasonic probe 101 is canceled or alleviated.

In the above embodiment, robot 20 is configured as a seven-axis articulated robot capable of translational movement in three directions and rotational movement in three directions. However, the number of axes may be any number. Robot 20 may be configured by a so-called vertical articulated robot, a horizontal articulated robot, or the like.

In the above-described embodiment, ultrasonic diagnostic system 10 includes robot 20 that automatically operates in accordance with a task program. However, the ultrasonic diagnostic system may include a master device installed at a remote location and operable by an operator, and a remote control robot connected to the master device via a communication line and holding the ultrasonic probe in the arm and operating the arm in accordance with the operation of the master device.

As described above, in the ultrasonic diagnostic system of the present disclosure, when a specific pattern is designated as the movement pattern of the probe, the control device cancels or alleviates the pressing of the probe against the body surface of the subject, and then moves the probe with the specific pattern. Then, when the movement with the specific pattern is ended, the control device presses the probe against the body surface. By determining a movement pattern that easily places a burden on the subject as a specific pattern, it is possible to reduce the burden on the subject during ultrasonic diagnosis.

In addition, in the ultrasonic diagnostic system of the present disclosure, the following configuration can be adopted. In other words, in the ultrasonic diagnostic system of the present disclosure, the specific pattern may be a pattern for rotating the probe about an axis or a pattern for moving the probe by a predetermined distance or more.

Further, in the ultrasonic diagnostic system of the present disclosure, a force sensor for detecting a reaction force acting on the probe may be provided, and the control device may control the moving device as the specific pattern control such that the pressing against the body surface is canceled or alleviated based on the reaction force detected by the force sensor. In this manner, it is possible to appropriately cancel or alleviate the pressing of the probe.

In addition, in the ultrasonic diagnostic system of the present disclosure, a position measurement sensor for measuring a position of the probe may be provided, and the control device may control the moving device as the specific pattern control such that the pressing against the body surface is canceled or alleviated based on a position measured by the position measurement sensor. In this manner, it is possible to appropriately cancel or alleviate the pressing of the probe.

In addition, in the ultrasonic diagnostic system of the present disclosure, the moving device may move the probe by driving a motor based on a torque command value, and the control device may control the moving device as the specific pattern control such that the pressing against the body surface is canceled or alleviated by reducing the torque command value. In this way, it is possible to appropriately cancel or alleviate the pressing of the probe without using the sensor.

In addition, in the ultrasonic diagnostic system of the present disclosure, the control device may control the moving device as the specific pattern control such that the pressing against the body surface is canceled or alleviated based on a change in an ultrasonic image acquired from the ultrasonic diagnostic device. In this way, it is possible to appropriately cancel or alleviate the pressing of the probe without using the sensor.

In addition, in the ultrasonic diagnostic system of the present disclosure, the moving device may be an arm robot having an articulated arm, and the probe may be attached to a tip end portion of the articulated arm.

A second ultrasonic diagnostic system of the present disclosure includes an ultrasonic diagnostic device having a probe, a moving device for moving the probe, a force sensor for detecting a reaction force acting on the probe, and a control device for controlling the moving device such that the probe is pressed against the body surface of a subject based on the reaction force detected by the force sensor when ultrasonic diagnosis is performed on the subject, or such that the pressing of the probe against the body surface is canceled or alleviated.

A third ultrasonic diagnostic system of the present disclosure includes an ultrasonic diagnostic device having a probe, a moving device for moving the probe, a position measurement sensor for measuring the position of the probe, and a control device for controlling the moving device such that the probe is pressed against the body surface of a subject based on a position measured by the position measurement sensor or such that the pressing of the probe against the body surface is canceled or alleviated when performing ultrasonic diagnosis on the subject.

### INDUSTRIAL APPLICABILITY

The present disclosure can be applied to the manufacturing industry of an ultrasonic diagnostic system and the like.

### REFERENCE SIGNS LIST

10: ultrasonic diagnostic system, 20, 120: robot, 21: first arm, 22: second arm, 25: base, 26: base plate, 28: force sensor, 31: first joint shaft, 32: second joint shaft, 33: orientation holding shaft, 35: first arm driving device, 35a: motor, 35b: encoder, 36: second arm driving device, 36a: motor, 36b: encoder, 37: orientation holding device, 37a: motor, 37b: encoder, 40: lifting and lowering device, 41: slider, 42: guide member, 43: ball screw shaft, 44: motor, 45: encoder, 50: three-axis rotation mechanism, 51: first rotation shaft, 52: second rotation shaft, 53: third rotation shaft, 55: first rotation device, 55a: motor, 55b: encoder, 56: second rotation device, 56a: motor, 56b: encoder, 57: third rotation device, 57a: motor, 57b: encoder, 60: holder, 70: control device, 71: CPU, 72: ROM, 73: RAM, 100: ultrasonic diagnostic device, 101: ultrasonic probe, 101a: cable, 102: ultrasonic diagnostic device main body, 103: control section, 104: instruction input section, 105: image processing section, 106: display section, 128 position measurement sensor

1. An ultrasonic diagnostic system comprising:
an ultrasonic diagnostic device having a probe;
a moving device configured to move the probe; and
a control device configured to control the moving device such that the probe is pressed against a body surface of a subject, in a case where a specific pattern is designated as a movement pattern of the probe, control the moving device such that the pressing of the probe against the body surface is canceled or alleviated, then control the moving device such that the probe is moved with the specific pattern, and when the movement with the specific pattern is ended, perform a specific pattern control for controlling the moving device such that the probe is pressed against the body surface, when ultrasonic diagnosis is performed on the subject.

2. The ultrasonic diagnostic system according to claim 1, wherein
the specific pattern is a pattern for rotating the probe about an axis or a pattern for moving the probe by a predetermined distance or more.

3. The ultrasonic diagnostic system according to claim 1, further comprising:
a force sensor configured to detect a reaction force acting on the probe, wherein
the control device controls the moving device such that the pressing against the body surface is canceled or alleviated based on the reaction force detected by the force sensor as the specific pattern control.

4. The ultrasonic diagnostic system according to claim 1, further comprising:
a position measurement sensor configured to measure a position of the probe, wherein
the control device controls the moving device such that the pressing against the body surface is canceled or alleviated based on the position measured by the position measurement sensor as the specific pattern control.

5. The ultrasonic diagnostic system according to claim 1, wherein
the moving device moves the probe by driving a motor based on a torque command value, and
the control device controls the moving device such that the pressing against the body surface is canceled or alleviated by reducing the torque command value as the specific pattern control.

6. The ultrasonic diagnostic system according to claim 1, wherein
the control device controls the moving device such that the pressing against the body surface is canceled or alleviated based on a change in an ultrasonic image acquired from the ultrasonic diagnostic device as the specific pattern control.

7. The ultrasonic diagnostic system according to claim 1, wherein
the moving device is an arm robot having an articulated arm, and
the probe is attached to a tip end portion of the articulated arm.

8. An ultrasonic diagnostic system comprising:
an ultrasonic diagnostic device having a probe;
a moving device configured to move the probe;
a force sensor configured to detect a reaction force acting on the probe; and
a control device configured to control the moving device such that the probe is pressed against a body surface of a subject based on the reaction force detected by the force sensor, or such that the pressing of the probe against the body surface is canceled or alleviated when ultrasonic diagnosis is performed on the subject.

9. An ultrasonic diagnostic system comprising:
an ultrasonic diagnostic device having a probe;
a moving device configured to move the probe;
a position measurement sensor configured to measure a position of the probe; and
a control device configured to control the moving device such that the probe is pressed against a body surface of a subject based on the position measured by the position measurement sensor, or such that the pressing of the probe against the body surface is canceled or alleviated when ultrasonic diagnosis is performed on the subject.
